(19) United States
(12) Patent Application Publication
(10) Pub. No.: US 2025/0249205 A1
(43) Pub. Date: Aug. 7, 2025

(54) CATHETER

(71) Applicant: TERUMO KABUSHIKI KAISHA, Tokyo (JP)

(72) Inventors: Takashi ITO, Shizuoka-shi (JP); Mitsuki HOSHI, Irvine, CA (US); Arisa TAKAYAMA, Fujinomiya-shi (JP)

(73) Assignee: TERUMO KABUSHIKI KAISHA, Tokyo (JP)

(21) Appl. No.: 19/186,903

(22) Filed: Apr. 23, 2025

Related U.S. Application Data
(63) Continuation of application No. PCT/JP2023/035137, filed on Sep. 27, 2023.

(30) Foreign Application Priority Data
Oct. 25, 2022 (JP) 2022-170336

(51) Int. Cl. A61M 25/00; A61M 25/01; A61M 25/06

(52) U.S. Cl. CPC A61M 25/0023; A61M 25/0045; A61M 25/0068; A61M 25/0108; A61M 25/0662

(57) ABSTRACT

A catheter includes an elongated shaft having a lumen communicating from a distal end to a proximal end and having a distal end tip at the distal end, in which the shaft has a tapered portion whose outer diameter gradually decreases in a tapered shape toward the distal end on an outer peripheral surface on a distal end side including at least the distal end tip, the tapered portion has a distal end tapered portion at a distal-most end and an intermediate tapered portion arranged on a proximal end side of the distal end tapered portion and having an inclination angle with respect to an axial center of the shaft smaller than the inclination angle of the distal end tapered portion, and a length of the intermediate tapered portion along the axial center is larger than a minimum inner diameter of the distal end tip.

### CROSS-REFERENCES TO RELATED APPLICATIONS

This application is a continuation of International Application No. PCT/JP2023/035137 filed on Sep. 27, 2023, which claims priority to Japanese Patent Application No. 2022-170336 filed on Oct. 25, 2022, the entire content of both of which is incorporated herein by reference.

### TECHNOLOGICAL FIELD

The present invention generally relates to a catheter used in a lumen of a blood vessel or the like.

### BACKGROUND DISCUSSION

In recent years, intraluminal treatment of a blood vessel or the like using a catheter has been actively performed because surgical stress is very low. For example, a catheter that is selectively introduced into and used in a branched blood vessel that is branched in a complicated manner in a body is generally pushed along a guide wire that is introduced into the blood vessel in advance to circulate a therapeutic medical agent, a diagnostic contrast agent, or the like through a lumen.

Meanwhile, a support catheter is used to support passage of a guide wire through a stenosed lesion (see, for example, JP 2019-503777 A). After the guide wire passes through the lesion, it is also necessary to pass the support catheter itself through the lesion to replace the guide wire or pre-expand the lesion before passing a balloon through the lesion. In order to allow the support catheter to enter the lesion, it is preferable that the distal-most end of the support catheter be hardly deformed and that the outer diameter of the distal end of the support catheter be thin. Therefore, a tapered portion whose outer diameter decreases in a tapered shape toward the distal end is preferably formed at the distal-most end of the support catheter.

### SUMMARY

If the length of a tapered portion at a distal end of a catheter along the axial center is too long, a thin region having a small outer diameter is formed widely at the distal end of the catheter. Thus, deformation easily occurs. Further, because the thickness of the distal end portion of the catheter decreases, it tends to be difficult to arrange a radiopaque marker in the vicinity of the distal end of the catheter. However, if the length of the tapered portion at the distal end of the catheter along the axial center is too short, an inclination of the tapered portion with respect to the axial center increases, and lesion crossability (the ability to extend across a lesion) deteriorates. Therefore, it is desirable that the catheter is configured so that the tapered portion at the distal end of the catheter is not so easily deformed and exhibits high crossability.

The catheter disclosed here has a tapered portion provided at a distal end that is hardly deformed and has high crossability.
(1) A catheter disclosed here includes an elongated shaft having a lumen communicating from a distal end to a proximal end and having a distal end tip at the distal end, in which the shaft has a tapered portion whose outer diameter gradually decreases in a tapered shape toward the distal end on an outer peripheral surface on a distal end side including at least the distal end tip, the tapered portion has a distal end tapered portion at a distal-most end and an intermediate tapered portion arranged on a proximal end side of the distal end tapered portion and having an inclination angle with respect to an axial center of the shaft smaller than the inclination angle of the distal end tapered portion, and a length of the intermediate tapered portion along the axial center is larger than a minimum inner diameter of the distal end tip.

Because the catheter according to (1) has the distal end tapered portion having a larger inclination angle than the intermediate tapered portion, the shaft can have the outer diameter and the thickness that are increased in a short range from the distal-most end toward the proximal end while having a small outer diameter at the most distal end. Therefore, the shaft is hardly deformed by reducing a portion having a small thickness while thinning the distal-most end. The shaft has the thin distal-most end and thus has high crossability. Further, because the length of the intermediate tapered portion along the axial center is larger than the minimum inner diameter of the distal end tip, the length is larger than the outer diameter of a guide wire insertable into the lumen. This makes it possible to suppress a rapid increase in the outer diameter in the intermediate tapered portion. Therefore, the intermediate tapered portion easily enters a narrow region and can have high crossability. Accordingly, the present catheter can have the tapered portion provided at the distal end which is hardly deformed and have high crossability.
(2) In the catheter according to (1), the tapered portion may have the distal end tapered portion, the intermediate tapered portion adjacent to a proximal end of the distal end tapered portion, and a proximal end tapered portion adjacent to a proximal end of the intermediate tapered portion and having an inclination angle with respect to the axial center of the shaft smaller than the inclination angle of the intermediate tapered portion, and the catheter may be a support catheter for supporting passage of a guide wire through the lumen. Therefore, because the catheter is pushed into a lesion along the guide wire passing through the lumen, the catheter can effectively enter a narrow lesion with the tapered portion that is hardly deformed and has high crossability.
(3) In the catheter according to (1) or (2), inclination angles of all outer peripheral surfaces of the tapered portion with respect to the axial center of the shaft may exceed 0 degrees. Therefore, because there is no region having a constant outer diameter along the axial center in the tapered portion (i.e., the outer diameter of the tapered portion continuously tapers), it is possible to suppress the length of the tapered portion along the axial center from becoming too long. Therefore, in the catheter, it is possible to suppress a region having a small outer diameter from being widely formed at the distal end portion of the catheter and to make the distal end portion of the catheter less susceptible to deformation. Further, because the catheter can suppress the length of the tapered portion along the axial center from becoming too long, it is easy to secure the thickness of the distal end portion of the catheter and arrange a radiopaque marker in the vicinity of the distal end of the catheter.
(4) In the catheter according to any one of (1) to (3), the minimum inner diameter of the distal end tip may be an inner diameter of the distal-most end of the shaft. This makes it possible to suppress the thickness of the distal end of the catheter from becoming too thin and to make the distal end of the catheter less susceptible to deformation.
(5) In the catheter according to any one of (1) to (4), the shaft may have a shaft main body connected to a proximal end of the distal end tip, and the distal end tip may be made from a material different than the shaft main body. Therefore, the catheter can have a wide selection range of the material of the distal end tip and have the tapered portion that is hardly deformed and has high crossability.
(6) In the catheter according to any one of (1) to (5), the shaft may include a radiopaque marker, and a distal end of the marker may be arranged on a distal end side of the intermediate tapered portion rather than a proximal end of the intermediate tapered portion. Therefore, in the catheter, the radiopaque marker can be arranged in the vicinity of the distal end of the catheter. Further, because the thickness of the proximal end of the intermediate tapered portion is sufficiently secured, the marker can be embedded in the shaft.

According to another aspect, a catheter comprises an elongated shaft having an outer periphery that extends from a most distal end of the elongated shaft to a most proximal end of the elongated shaft, the elongated shaft including a tapered portion and a constant outer diameter portion. The tapered portion of the elongated shaft has a most distal end and a most proximal end at opposite ends of the tapered portion, and the constant outer diameter portion of the elongated shaft has a most distal end and a most proximal end at opposite ends of the constant outer diameter portion, and wherein the most proximal end of the tapered portion of the elongated shaft is connected to the most distal end of the constant outer diameter portion of the elongated shaft. The tapered portion of the elongated shaft has an outer diameter that tapers from the most distal end of the tapered portion toward the most proximal end of the tapered portion so that the outer diameter of the most distal end of the tapered portion is less than the outer diameter of the most proximal end of the tapered portion, and the constant outer diameter portion of the elongated shaft has an outer diameter that is constant from the most distal end of the constant outer diameter portion towards the most proximal end of the constant outer diameter portion. The elongated shaft includes an axially extending lumen that extends axially throughout the elongated shaft from the most distal end of the elongated shaft to the most proximal end of the elongated shaft so that the axially extending lumen passes through the tapered portion and through the constant outer diameter portion. The elongated shaft has an axial center and is comprised of an axially extending shaft main body and an axially extending distal end tip, with the axially extending distal end tip being on a distal side of the axially extending shaft main body so that a distal end of the axially extending shaft main body is connected to a proximal end of the axially extending distal end tip. The tapered portion of the elongated shaft is defined by an entirety of the axially extending distal end tip and a most distal portion of the axially extending shaft main body. The entirety of the outer peripheral surface of the axially extending distal end tip is exposed, and the entirety of the inner peripheral surface of the axially extending distal end tip is exposed. The tapered portion of the elongated shaft includes a distal end tapered portion and an intermediate tapered portion, with the distal end tapered portion being at the most distal end of the tapered portion, and the intermediate tapered portion being on a proximal side of the distal end tapered portion. The distal end tapered portion and the intermediate tapered portion are each tapered so that an outer surface of the distal end tapered portion and the outer surface of the intermediate tapered portion are inclined at an inclination angle with respect to the axial center of the shaft, with the inclination angle of an entirety of the intermediate tapered portion being smaller than the inclination angle of an entirety of the distal end tapered portion, and the length of the intermediate tapered portion along the axial center being larger than the minimum inner diameter of the distal end tip of the elongated shaft.

In accordance with another aspect, a catheter comprises an elongated shaft having an outer periphery that extends from a most distal end of the elongated shaft to a most proximal end of the elongated shaft and a hub fixed to a proximal end of the elongated shaft, wherein the elongated shaft has an axial center, and the elongated shaft includes an axially extending shaft main body and an axially extending distal end tip. The elongated shaft includes an axially extending lumen that extends axially throughout the elongated shaft from the most distal end of the elongated shaft to the most proximal end of the elongated shaft so that the axially extending lumen passes through the axially extending distal end tip and through the axially extending shaft main body. The axially extending shaft main body includes an axially extending inner layer and an axially extending outer layer, with a most distal portion of the axially extending inner layer and a most distal portion of the axially extending outer layer axially overlapping one another, and the most distal portion of the axially extending inner layer and the most distal portion of the axially extending outer layer axially overlapping a most proximal portion of the distal end tip. The axially extending distal end tip has a most distal end and a most proximal end at opposite ends of the axially extending distal end tip, with the axially extending distal end tip of the elongated shaft having an outer diameter that tapers from the most distal end of the axially extending distal end tip to the most proximal end of the axially extending distal end tip so that the outer diameter of the most distal end of the axially extending distal end tip is less than the outer diameter of the most proximal end of the axially extending distal end tip. The elongated shaft includes an axially extending lumen that extends axially throughout the elongated shaft from the most distal end of the elongated shaft to the most proximal end of the elongated shaft so that the axially extending lumen passes through the axially extending distal end tip and through the axially extending shaft main body. The axially extending distal end tip of the elongated shaft includes a distal end tapered portion and an intermediate tapered portion, with the distal end tapered portion being at the most distal end of the axially extending distal end tip, and the intermediate tapered portion being on a proximal side of the distal end tapered portion. The distal end tapered portion and the intermediate tapered portion are each tapered so that an outer surface of the distal end tapered portion and the outer surface of the intermediate tapered portion are inclined at an inclination angle with respect to the axial center of the shaft, with the inclination angle of an entirety of the intermediate tapered portion being smaller than the inclination angle of an entirety of the distal end tapered portion. In addition, the length of the intermediate tapered portion along the axial center is larger than a minimum inner diameter of the distal end tip of the elongated shaft.

### BRIEF DESCRIPTION OF THE DRAWINGS

FIG. 1 is a plan view of a catheter according to an embodiment.

FIG. 2 is a plan view of a distal end portion of the catheter according to the embodiment.

FIG. 3 is a cross-sectional view of the distal end portion of the catheter according to the embodiment.

### DETAILED DESCRIPTION

Set forth below with reference to the accompanying drawings is a detailed description of an embodiment of a catheter, representing an example of the new catheter disclosed here. The dimensions or scales on the drawings may be exaggerated or different from actuality/reality for convenience of description and illustration. In the present specification and the drawings, components having substantially the same functional configuration are denoted by the same reference numerals, and a detailed description of already described features is not repeated. In the present specification, a side of a catheter to be inserted into a body lumen is referred to as a “distal end side”, and a side to be operated is referred to as a “proximal end side”.

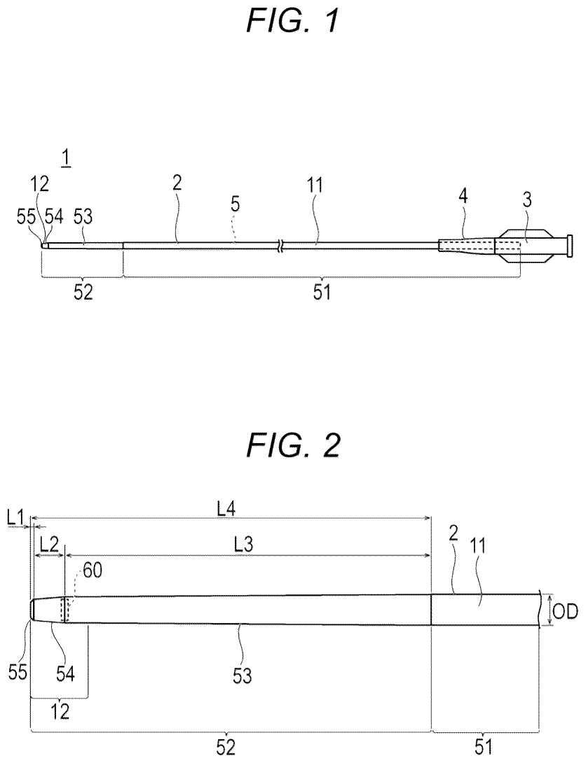

A catheter 1 according to an embodiment is used for performing treatment, diagnosis, and the like by, for example, introducing the catheter into a blood vessel from the radial artery of an arm, the femoral artery of a foot, or the lower extremity peripheral artery, followed by inserting the catheter into, for example, the artery of a lower limb, and passing the catheter through a stenosed portion of the artery of the lower limb or chronic total occlusion (CTO). A treatment target portion is not limited to the artery of the lower limb. The catheter 1 is, for example, the support catheter 1, but may be a device for other purposes. The artery of the lower limb is an artery in the vicinity of and distal to the aortoiliac bifurcation and collateral flow. As illustrated in FIG. 1, the catheter 1 includes a long or elongated shaft 2, a hub 3 connected to the proximal end of the shaft 2, and a strain relief 4 provided at a connection portion between the shaft 2 and the hub 3.

As illustrated in FIGS. 1 to 3, the shaft 2 is a tubular member having flexibility and has a lumen 5 formed inside and extending from the proximal end (most proximal end) of the shaft to the distal end (most distal end) of the shaft. A guide wire is inserted through (positioned in) the lumen 5 when the catheter 1 is inserted into a blood vessel. The lumen 5 can also be used as a passage for medicinal solutions, embolic materials, contrast agents, medical instruments, or the like.

The effective length of the shaft 2 is not particularly limited and is appropriately set depending on a blood vessel into which the shaft 2 is to be inserted. For example, the effective length is 200 mm to 2600 mm. The effective length of the shaft 2 is a length of a portion insertable into a blood vessel, a sheath, or the like. In the present embodiment, the effective length is a length from the distal-most end of the strain relief 4 to the distal-most end of the shaft 2.

The shaft 2 has a shaft main body 11, a distal end tip 12 connected to the distal end of the shaft main body 11, and a radiopaque marker 60. The shaft main body 11 includes a plurality of layers and includes an inner layer 20 forming an inner surface of the lumen 5, a reinforcing body 30 arranged radially outside the inner layer 20, and an outer layer 40 formed radially outside the inner layer 20 and the reinforcing body 30. The term “radially” matches with or refers to a radial direction from the axial center of the shaft 2. The “radially outside” is a side away from the axial center of the shaft 2 in the radial direction. The shaft main body 11 has a distal end surface 13 connected to the distal end tip 12 at a distal end portion, a marker arrangement portion 14 formed with a constant outer diameter from the distal end surface 13 toward the proximal end direction and on which the marker 60 is arranged on the radial outside, and a main body tapered portion 15 having an outer diameter that increases in a tapered shape from the proximal end of the marker arrangement portion 14 toward the proximal end direction and connected to the distal end tip 12.

The lumen 5 is formed inside the inner layer 20.

A material of the inner layer 20 can be a thermoplastic resin, a thermosetting resin, or the like and is preferably a fluorine-based resin such as polytetrafluoroethylene (PTFE), a low-friction material such as high-density polyethylene (HDPE), or the like, but may be a polyamide resin, a polyamide elastomer, polyester, a polyester elastomer, or the like.

The reinforcing body 30 is formed on the outer periphery of the inner layer 20 by braiding a plurality of wire materials in a tubular shape to have a gap. In the reinforcing body 30, the wire materials may be horizontally wound in the same direction or may be wound while a winding direction is changed, for example, between clockwise or counterclockwise. Further, a winding pitch, a lattice distance, an inclination angle with respect to a circumferential direction, or the like may be changed depending on a position. That is, the configuration is not particularly limited. For example, one wire material may be wound in a coil shape in one direction.

The reinforcing body 30 may be arranged on the entire shaft 2 along an axial center X or may be arranged only on a part thereof. In the present embodiment, the reinforcing body 30 is arranged on the proximal end side of the shaft main body 11 rather than the distal end thereof and is not arranged on the distal end tip 12. A portion at the distal end portion of the shaft 2 where the reinforcing body 30 is not arranged has high flexibility, and thus a bending direction can be easily changed along the guide wire. Further, a burden on a living tissue with which the portion comes in contact can be reduced.

The wire diameter (diameter) of the wire material of the reinforcing body 30 is not particularly limited, but is, for example, 0.03 mm to 0.08 mm. As an example, the wire diameter is 0.03 mm.

The wire material of the reinforcing body 30 can be a metal wire made from stainless steel, platinum (Pt), tungsten (W), or the like, a resin fiber, a carbon fiber, a glass fiber, or the like. Alternatively, a plurality of those wire materials may be used in combination. The wire material may be a round wire, an elliptic wire, or a flat wire as seen in a transverse cross-section. Each wire material may be used as one wire for braiding, or two or more wire materials may be bundled and used as one wire for braiding. For example, two wires may be used.

The outer layer 40 is a tubular body arranged on the outer periphery of the inner layer 20 and the reinforcing body 30.

Examples of a material of the outer layer 40 include a polymer material such as polyolefin (for example, polyethylene, polypropylene, polybutene, an ethylene-propylene copolymer, an ethylene-vinyl acetate copolymer, an ionomer, or a mixture of two or more types thereof), polyvinyl chloride, polyamide, a polyester elastomer, a polyamide elastomer, polyurethane, a polyurethane elastomer, polyimide, or a fluororesin, a thermoplastic resin such as a mixture thereof, and a thermosetting resin such as an epoxy resin. The outer layer 40 may be mixed with a radiopaque substance, and the material of the outer layer 40 may be the same as the material of the inner layer 20. As an example, a resin and a radiopaque substance may be mixed.

The structure of the shaft main body 11 is not limited thereto. For example, the shaft main body 11 does not need to have the main body tapered portion 15. The distal-most end of the shaft main body 11 includes the inner layer 20 and the outer layer 40, but may include the inner layer 20 or the outer layer 40.

The marker 60 is a radiopaque (radiographic contrast) member arranged radially outside the marker arrangement portion 14 of the shaft main body 11. The number of the markers 60 is one in the present embodiment, but may be two or three or more. The marker 60 is a member formed by covering the outside of the inner layer 20 with a member that is made from a radiopaque material and whose cross-sectional shape orthogonal to the axial center X is a pipe or tube (i.e., a tubular body), then crimping the member, and forming the member into a tubular shape. However, the marker 60 is not limited thereto. For example, the marker 60 may be formed by winding a wire or a belt-shaped member made from a radiopaque material in a coil shape or may have a C-shape. A position of the shaft 2 where the marker 60 is arranged is not limited. For example, the marker 60 does not need to be arranged radially outside the marker arrangement portion 14 and may be embedded in the outer layer 40 of the marker arrangement portion 14, for example. Alternatively, the marker 60 does not need to be arranged.

Examples of a material that can be preferably used for the marker 60 include a material obtained by kneading radiopaque materials such as platinum, gold, silver, tungsten, iridium, molybdenum, tantalum, an alloy thereof, or a metal powder thereof, barium sulfate, bismuth oxide, or a coupling compound thereof. Further, a metal coil or solder having the contrast property may be used. As an example, a platinum-iridium alloy is used.

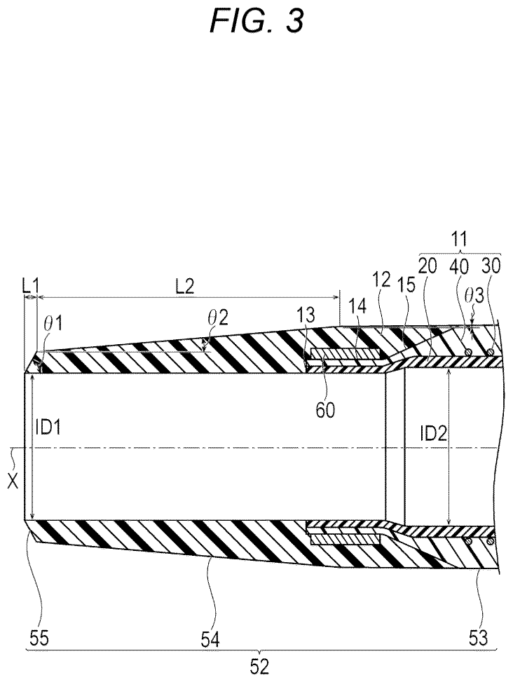

The distal end tip 12 is a member connected to the distal end of the shaft main body 11 having the inner layer 20, the reinforcing body 30, and the outer layer 40. A proximal end surface of the distal end tip 12 is connected to the distal end surface 13 and the main body tapered portion 15 of the shaft main body 11 and covers the radial outside of the marker 60 arranged radially outside the marker arrangement portion 14 of the shaft main body 11. As illustrated in FIG. 3, the entirety of the outer surface of the distal end tip 12 is exposed (not covered). Similarly, the entirety of the inner surface of the distal end tip 12 is exposed (not covered).

On an outer peripheral surface of the shaft 2, an outer diameter constant portion (constant outer diameter portion) 51 having a constant outer diameter along the axial center X (axial direction) and the tapered portion 52 arranged on the distal end side of the outer diameter constant portion 51 and having the outer diameter that gradually decreases in a tapered shape toward the distal end are formed. In the present embodiment, the tapered portion 52 includes a predetermined range on the distal end side of the shaft main body 11 and the distal end tip 12. The tapered portion 52 does not need to include the shaft main body 11, but may include only the distal end tip 12. The tapered portion 52 has a proximal end tapered portion 53 connected to the distal end of the outer diameter constant portion 51, an intermediate tapered portion 54 connected to the distal end of the proximal end tapered portion 53, and a distal end tapered portion 55 connected to the distal end of the intermediate tapered portion 54. A intermediate inclination angle θ2, which is an inclination angle of the outer peripheral surface of the intermediate tapered portion 54 with respect to the axial center X of the shaft 2, is larger than a proximal end inclination angle θ3, which is an inclination angle of the outer peripheral surface of the proximal end tapered portion 53 with respect to the axial center X of the shaft 2. A distal end inclination angle θ1, which is an inclination angle of the outer peripheral surface of the distal end tapered portion 55 with respect to the axial center X of the shaft 2, is larger than the intermediate inclination angle θ2 of the intermediate tapered portion 54. The distal end inclination angle θ1, the intermediate inclination angle θ2, and the proximal end inclination angle θ3 are inclination angles with respect to the axial center X, but in FIG. 3, for convenience, those angles will be described as inclination angles with respect to a virtual line parallel to the axial center X.

A length L2 of the intermediate tapered portion 54 along the axial center X (axial direction) is longer than a length L1 of the distal end tapered portion 55 along the axial center X (axial direction). A length L3 of the proximal end tapered portion 53 along the axial center X (axial direction) is longer than the length L2 of the intermediate tapered portion 54 along the axial center X. The length L1 of the distal end tapered portion 55 is not particularly limited, but is, for example, 0.06 mm. The length L2 of the intermediate tapered portion 54 is not particularly limited, but is preferably 0.52 mm to 1.2 mm, and more preferably 0.8 mm to 1.2 mm. The length L3 of the proximal end tapered portion 53 is not particularly limited, but is preferably 7.2 mm to 10.8 mm. The length L4 of the entire tapered portion 52 along the axial center X (axial direction) is not particularly limited, but is, for example, 12 mm.

An outer diameter OD of the outer diameter constant portion 51 is not particularly limited, but is, for example, 0.75 mm. An inner diameter (minimum inner diameter) ID1 of a portion of the shaft 2 having the smallest inner diameter is not particularly limited, but is, for example, 0.42 mm. In the present embodiment, the inner diameter of a portion where the distal end tapered portion 55 of the shaft 2 is formed and the inner diameter of a portion where at least the distal end portion of the intermediate tapered portion 54 is formed match with (i.e., are the same as) the minimum inner diameter ID1 of the shaft 2. An inner diameter ID2 on the proximal end side of the shaft 2 is not particularly limited, but is larger than the inner diameter ID1 and is, for example, 0.48 mm. In the present embodiment, the inner diameter of a portion where at least the proximal end portion of the proximal end tapered portion 53 of the shaft 2 is formed and the inner diameter of a portion where the outer diameter constant portion is formed match with the inner diameter ID2. An inner peripheral surface in which the inner diameter ID1 of the shaft 2 is formed and an inner peripheral surface in which the inner diameter ID2 is formed are preferably formed to have an inner peripheral surface whose inner diameter decreases in a tapered shape toward the distal end.

The tapered portion 52 does not have a portion having a constant outer diameter along the axial center X. At all positions of the outer peripheral surface of the tapered portion 52, the inclination angle with respect to the axial center of the shaft 2 exceeds 0 degrees such that the outer diameter increases toward the proximal end or in the proximal direction.

The distal end inclination angle θ1 of the distal end tapered portion 55 is larger than the intermediate inclination angle θ2 and the proximal end inclination angle θ3, and the length L1 of the distal end tapered portion 55 along the axial center X (axial direction) is shorter than the length L2 of the intermediate tapered portion 54 and the length L3 of the proximal end tapered portion 53. Thus, the shaft 2 can have the outer diameter and the thickness that are rapidly increased in a short range from the distal-most end toward the proximal end or in the proximal direction while having a small outer diameter at the distal-most end. Therefore, the shaft 2 has high crossability because of a thin distal-most end, and the tapered portion 52 is hardly deformed because a portion having a small thickness is reduced.

The length L2 of the intermediate tapered portion 54 along the axial center X (axial direction) is longer than the inner diameter ID1 of the intermediate tapered portion 54. The inner diameter ID1 substantially matches with the maximum outer diameter of a guide wire that can pass through the lumen 5, and thus the length L2 of the intermediate tapered portion 54 along the axial center X (axial direction) is larger than the maximum outer diameter of the guide wire that passes through the lumen 5. A ratio A=L2/ID1 of the length L2 to the inner diameter ID1 is not particularly limited, but is larger than 1, and is preferably 1.9 to 2.9.

A ratio B=L2/ID2 of the length L2 to the inner diameter ID2 is not particularly limited, but is preferably 1.6 to 2.5.

A ratio C=L2/OD of the length L2 to the outer diameter OD of the outer diameter constant portion 51 is not particularly limited, but is preferably 0.65 to 1.6.

The material of the distal end tip 12 can be the above-described materials applicable to the outer layer 40, for example. The distal end tip 12 may be integrally formed as the same structure by a mold with the same material as those of the outer layer 40 and/or the inner layer 20 of the shaft main body 11. Alternatively, the distal end tip 12 may be retrofitted or joined to the shaft main body 11 with a different material than the outer layer 40.

The proximal end portion of the shaft 2 is liquid-tightly fixed to the hub 3 with an adhesive, by heat fusion, or with a stopper (not illustrated), for example. The hub 3 functions as, for example, an insertion port for a guide wire or a medical instrument into the lumen 5 or an injection port for a medicinal solution, an embolic material, a contrast agent, or the like into the lumen 5 and also functions as a grip portion when the catheter 1 is operated. The material of the hub 3 is not particularly limited, and examples of materials that can be preferably used include thermoplastic resins such as polycarbonate, polyamide, polysulfone, polyarylate, or a methacrylate-butylene-styrene copolymer.

The strain relief 4 is made from an elastic material provided to surround the shaft 2 and suppresses kinking of the shaft 2 at the connection portion between the shaft 2 and the hub 3. Examples of materials that can be preferably used for the strain relief 4 include natural rubber, silicone resin, polyester elastomer, polyamide elastomer, and polyurethane elastomer.

As described above, the catheter 1 according to the present embodiment is the catheter 1 including the long (elongated) shaft 2 having the lumen 5 communicating from the distal end to the proximal end and having the distal end tip 12 at the distal end, in which the shaft 2 has the tapered portion 52 whose outer diameter gradually decreases in a tapered shape toward the distal end on an outer peripheral surface on the distal end side including at least the distal end tip 12, the tapered portion 52 has the distal end tapered portion 55 at the distal-most end and the intermediate tapered portion 54 arranged on the proximal end side of the distal end tapered portion 55 and having the inclination angle with respect to the axial center X of the shaft 2 smaller than the inclination angle of the distal end tapered portion 55, and the length L2 of the intermediate tapered portion 54 along the axial center X (axial direction) is larger than the minimum inner diameter ID1 of the distal end tip 12. Therefore, because the catheter 1 has the distal end tapered portion 55 having a larger inclination angle than the intermediate tapered portion 54, the shaft 2 can have the outer diameter and the thickness that are increased in a short range from the distal-most end toward the proximal end while having a small outer diameter at the most distal end. Accordingly, the shaft 2 is hardly deformed by reducing a portion having a small thickness while thinning the distal-most end. The shaft 2 has a thin distal-most end and thus has high crossability. Further, because the length L2 of the intermediate tapered portion 54 along the axial center X is larger than the inner diameter ID1, the length is larger than the outer diameter of a guide wire insertable into the lumen 5. This makes it possible to suppress a rapid increase in the outer diameter in the intermediate tapered portion 54. Therefore, the intermediate tapered portion 54 easily enters a narrow region and can have high crossability. Accordingly, the present catheter 1 can have the tapered portion 52 provided at the distal end which is hardly deformed and have high crossability. The length L2 of the intermediate tapered portion 54 is larger than the outer diameter of the guide wire insertable into the lumen 5, whereas the length L1 of the distal end tapered portion 55 is smaller than the outer diameter of the guide wire insertable into the lumen 5. Therefore, the shaft 2 can have the outer diameter and the thickness that can be effectively increased in a short range of the length L1 from the distal-most end toward the proximal end while having a small outer diameter at the distal-most end.

The tapered portion 52 has the distal end tapered portion 55, the intermediate tapered portion 54 adjacent to a proximal end of the distal end tapered portion 55, and the proximal end tapered portion 53 adjacent to a proximal end of the intermediate tapered portion 54 and having the inclination angle with respect to the axial center X of the shaft 2 smaller than the inclination angle of the intermediate tapered portion 54; and the catheter 1 is a support catheter for supporting passage of a guide wire through the lumen 5. Therefore, because the catheter 1 is pushed into a lesion along the guide wire passing through the lumen 5, the catheter 1 can effectively enter a narrow lesion with the tapered portion 52 that is hardly deformed and has high crossability.

Inclination angles of all outer peripheral surfaces of the tapered portion 52 with respect to the axial center X of the shaft 2 exceed 0 degrees. Therefore, because there is no region having a constant outer diameter along the axial center X (axial direction) in the tapered portion 52, it is possible to suppress the length L4 of the tapered portion 52 along the axial center X from becoming too long. Therefore, in the catheter 1, it is possible to suppress a region having a small outer diameter from being widely formed at the distal end portion of the catheter 1 and to make the distal end portion of the catheter 1 less susceptible to deformation. Further, because the catheter 1 can suppress the length L4 of the tapered portion 52 along the axial center X from becoming too long, it is easy to secure the thickness of the distal end portion of the catheter 1 and arrange the radiopaque marker 60 in the vicinity of the distal end of the catheter 1.

The minimum inner diameter ID1 of the tapered portion 52 is the inner diameter of the distal-most end of the shaft 2. This makes it possible to suppress the thickness of the distal end of the catheter 1 from becoming too thin and to make the distal end of the catheter 1 less susceptible to deformation. The inner diameter of the intermediate tapered portion 54 may match with the minimum inner diameter ID1 of the tapered portion 52. This makes it possible to suppress the thickness of the intermediate tapered portion 54 of the catheter 1 from becoming too thin, and thus the radiopaque marker 60 is easily arranged in the vicinity of the distal end of the catheter 1.

The shaft 2 has the shaft main body 11 connected to the proximal end of the distal end tip 12, and the distal end tip 12 may be made from a material different than the shaft main body 11. Therefore, the catheter 1 can have a wide selection range of the material of the distal end tip 12 and have the tapered portion 52 having desirable characteristics (for example, hardly deformed and high crossability). For example, when the distal end tip 12 is made from a material softer than the material of the shaft main body 11, it is possible to reduce a burden on a living tissue that comes in contact with the distal end tip 12. When the distal end tip 12 is made from a material harder than the material of the shaft main body 11, the distal end tip 12 is further hardly deformed, and the crossability of the distal end tip 12 is further improved.

The shaft 2 has the radiopaque marker 60, and the distal end of the marker 60 is arranged on the distal end side of the intermediate tapered portion 54 rather than the proximal end (proximal end side) thereof. As shown in FIG. 3, the most distal end of the radiopaque marker 60 is arranged on the distal side of a most proximal end of the intermediate tapered portion 54. FIG. 3 also shows that the radiopaque marker 60 is positioned so that it straddles the most proximal end of the intermediate tapered portion 54, meaning a part of the radiopaque marker 60 is in the intermediate tapered portion 54 and a part of the intermediate tapered portion 54 is in the proximal end tapered portion 53. Therefore, in the catheter 1, the radiopaque marker 60 can be arranged in the vicinity of the distal end of the catheter 1. Further, because the thickness of the proximal end of the intermediate tapered portion 54 is sufficiently secured, the marker 60 can be embedded in the shaft 2.

The present invention is not limited to the embodiment described above, and those skilled in the art can make various modifications within the technical idea of the present invention. That is, the detailed description above describes embodiments of a catheter representing examples of the new catheter disclosed here. The invention is not limited, however, to the precise embodiment and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

### REFERENCE SIGNS LIST

1 Catheter
2 Shaft
3 Hub
4 Strain relief
5 Lumen
11 Shaft main body
12 Distal end tip
13 Distal end surface
14 Marker arrangement portion
15 Main body tapered portion
20 Inner layer
30 Reinforcing body
40 Outer layer
51 Outer diameter constant portion
52 Tapered portion
53 Proximal end tapered portion
54 Intermediate tapered portion
55 Distal end tapered portion
60 Marker
ID1 Minimum inner diameter of shaft
L1 Length of distal end tapered portion
L2 Length of intermediate tapered portion
L3 Length of proximal end tapered portion
θ1 Inclination angle of distal end tapered portion
θ2 Inclination angle of intermediate tapered portion
θ3 Inclination angle of proximal end tapered portion
X axial center

What is claimed is:

1. A catheter comprising:
an elongated shaft having a lumen communicating from a distal end of the elongated shaft to a proximal end of the elongated shaft and having a distal end tip at the distal end of the elongated shaft, the elongated shaft having an axial center,
the elongated shaft including a tapered portion whose outer diameter gradually decreases in a tapered shape toward the distal end of the elongated shaft, the tapered portion of the elongated shaft being on an outer peripheral surface of the elongated shaft and on a distal end side of the elongated shaft including at least the distal end tip of the elongated shaft,
the tapered portion of the elongated shaft including a distal end tapered portion and an intermediate tapered portion, the distal end tapered portion being at a distal-most end of the tapered portion, the intermediate tapered portion being on a proximal end side of the distal end tapered portion, the distal end tapered portion and the intermediate tapered portion each being tapered so that an outer surface of the distal end tapered portion and the outer surface of the intermediate tapered portion are inclined at an inclination angle with respect to the axial center of the shaft, the inclination angle of the intermediate tapered portion being smaller than the inclination angle of the distal end tapered portion, and
a length of the intermediate tapered portion along the axial center is larger than a minimum inner diameter of the distal end tip of the elongated shaft.

2. The catheter according to claim 1, wherein
the tapered portion includes, in addition to the distal end tapered portion and the intermediate tapered portion adjacent to a proximal end of the distal end tapered portion, a proximal end tapered portion adjacent to a proximal end of the intermediate tapered portion, the proximal end tapered portion being tapered so that an outer surface of the proximal end tapered portion is inclined at an inclination angle with respect to the axial center of the shaft, the inclination angle of the proximal end tapered portion being smaller than the inclination angle of the intermediate tapered portion, and
the catheter is a support catheter for supporting passage of a guide wire through the lumen.

3. The catheter according to claim 2, wherein the inclination angle of an entirety of the outer peripheral surface of the tapered portion with respect to the axial center of the shaft exceed 0 degrees.

4. The catheter of claim 2, wherein a minimum inner diameter of the distal end tip is an inner diameter of the distal-most end of the elongated shaft.

5. The catheter according to claim 2, wherein
the elongated shaft includes a shaft main body connected to a proximal end of the distal end tip, and
the distal end tip is made from a material different than the shaft main body.

6. The catheter according to claim 2, wherein
the shaft includes a radiopaque marker, and
a distal end of the radiopaque marker is on a distal end side of the intermediate tapered portion rather than a proximal end side of the intermediate tapered portion.

7. The catheter according to claim 1, wherein the inclination angle of an entirety of the outer peripheral surface of the tapered portion with respect to the axial center of the shaft exceed 0 degrees.

8. The catheter of claim 1, wherein a minimum inner diameter of the distal end tip is an inner diameter of the distal-most end of the elongated shaft.

9. The catheter according to claim 1, wherein
the elongated shaft includes a shaft main body connected to a proximal end of the distal end tip, and
the distal end tip is made from a material different than the shaft main body.

10. The catheter according to claim 1, wherein
the shaft includes a radiopaque marker, and
a distal end of the radiopaque marker is on a distal end side of the intermediate tapered portion rather than a proximal end side of the intermediate tapered portion.

11. A catheter comprising:
an elongated shaft having an outer periphery that extends from a most distal end of the elongated shaft to a most proximal end of the elongated shaft, the elongated shaft including a tapered portion and a constant outer diameter portion;
the tapered portion of the elongated shaft having a most distal end and a most proximal end at opposite ends of the tapered portion, the constant outer diameter portion of the elongated shaft having a most distal end and a most proximal end at opposite ends of the constant outer diameter portion, the most proximal end of the tapered portion of the elongated shaft being connected to the most distal end of the constant outer diameter portion of the elongated shaft;
the tapered portion of the elongated shaft having an outer diameter that tapers from the most distal end of the tapered portion toward the most proximal end of the tapered portion so that the outer diameter of the most distal end of the tapered portion is less than the outer diameter of the most proximal end of the tapered portion;
the constant outer diameter portion of the elongated shaft having an outer diameter that is constant from the most distal end of the constant outer diameter portion towards the most proximal end of the constant outer diameter portion;
the elongated shaft including an axially extending lumen that extends axially throughout the elongated shaft from the most distal end of the elongated shaft to the most proximal end of the elongated shaft so that the axially extending lumen passes through the tapered portion and through the constant outer diameter portion;
the elongated shaft having an axial center and being comprised of an axially extending shaft main body and an axially extending distal end tip, the axially extending distal end tip being on a distal side of the axially extending shaft main body so that a distal end of the axially extending shaft main body is connected to a proximal end of the axially extending distal end tip;
the tapered portion of the elongated shaft being defined by an entirety of the axially extending distal end tip and a most distal portion of the axially extending shaft main body;
an entirety of the outer peripheral surface of the axially extending distal end tip being exposed, an entirety of the inner peripheral surface of the axially extending distal end tip being exposed;
the tapered portion of the elongated shaft including a distal end tapered portion and an intermediate tapered portion, the distal end tapered portion being at the most distal end of the tapered portion, the intermediate tapered portion being on a proximal side of the distal end tapered portion, the distal end tapered portion and the intermediate tapered portion each being tapered so that an outer surface of the distal end tapered portion and the outer surface of the intermediate tapered portion are inclined at an inclination angle with respect to the axial center of the shaft, the inclination angle of an entirety of the intermediate tapered portion being smaller than the inclination angle of an entirety of the distal end tapered portion, and
a length of the intermediate tapered portion along the axial center is larger than a minimum inner diameter of the distal end tip of the elongated shaft.

12. The catheter of claim 11, wherein a most distal portion of the axially extending shaft main body includes an axially extending inner layer and an axially extending outer layer, the axially extending outer layer being positioned in overlying and axially overlapping relation to the axially extending inner layer.

13. The catheter of claim 12, wherein an entirety of the axially extending distal end tip located on a distal side of the axially extending inner and outer layers is made of the same material.

14. The catheter of claim 11, wherein a proximal portion of the axially extending distal end tip includes a radiopaque marker that is covered by material forming the axially extending distal end tip.

15. The catheter of claim 11, wherein the tapered portion of the elongated shaft includes, in addition to the distal end tapered portion and the intermediate tapered portion, a proximal end tapered portion, the proximal end tapered portion being on a proximal side of the intermediate tapered portion, the proximal end tapered portion being tapered so that an outer surface of the proximal end tapered portion is inclined at an inclination angle with respect to the axial center of the elongated, the inclination angle of an entirety of the proximal end tapered portion being smaller than the inclination angle of an entirety of the intermediate tapered portion.

16. The catheter of claim 11, wherein an entirety of the tapered portion of the elongated shaft, from the most distal end of the tapered portion to the most proximal end of the tapered portion, is devoid of a constant outer diameter portion having a constant outer diameter.

17. The catheter of claim 11, wherein the elongated shaft has an inner diameter, the inner diameter of the elongated shaft at a most distal portion of the tip end part is less than the inner diameter of the elongated shaft at a most proximal portion of the tip end part.

18. A catheter comprising:
an elongated shaft having an outer periphery that extends from a most distal end of the elongated shaft to a most proximal end of the elongated shaft and a hub fixed to a proximal end of the elongated shaft,
the elongated shaft having an axial center, the elongated shaft including an axially extending shaft main body and an axially extending distal end tip,
the elongated shaft including an axially extending lumen that extends axially throughout the elongated shaft from the most distal end of the elongated shaft to the most proximal end of the elongated shaft so that the axially extending lumen passes through the axially extending distal end tip and through the axially extending shaft main body,
the axially extending shaft main body including an axially extending inner layer and an axially extending outer layer, with a most distal portion of the axially extending inner layer and a most distal portion of the axially extending outer layer axially overlapping one another,
the most distal portion of the axially extending inner layer and the most distal portion of the axially extending outer layer axially overlapping a most proximal portion of the distal end tip,
the axially extending distal end tip having a most distal end and a most proximal end at opposite ends of the axially extending distal end tip, the axially extending distal end tip of the elongated shaft having an outer diameter that tapers from the most distal end of the axially extending distal end tip to the most proximal end of the axially extending distal end tip so that the outer diameter of the most distal end of the axially extending distal end tip is less than the outer diameter of the most proximal end of the axially extending distal end tip;
the elongated shaft including an axially extending lumen that extends axially throughout the elongated shaft from the most distal end of the elongated shaft to the most proximal end of the elongated shaft so that the axially extending lumen passes through the axially extending distal end tip and through the axially extending shaft main body;
the axially extending distal end tip of the elongated shaft including a distal end tapered portion and an intermediate tapered portion, the distal end tapered portion being at the most distal end of the axially extending distal end tip, the intermediate tapered portion being on a proximal side of the distal end tapered portion, the distal end tapered portion and the intermediate tapered portion each being tapered so that an outer surface of the distal end tapered portion and the outer surface of the intermediate tapered portion are inclined at an inclination angle with respect to the axial center of the shaft, the inclination angle of an entirety of the intermediate tapered portion being smaller than the inclination angle of an entirety of the distal end tapered portion, and
a length of the intermediate tapered portion along the axial center is larger than a minimum inner diameter of the distal end tip of the elongated shaft.

19. The catheter of claim 18, wherein an entirety of the outer peripheral surface of the axially extending distal end tip is exposed, and an entirety of the inner peripheral surface of the axially extending distal end tip is exposed.

20. The catheter of claim 18, wherein an entirety of the axially extending distal end tip of the elongated shaft, from the most distal end of the axially extending distal end tip to the most proximal end of the axially extending distal end tip, is devoid of a constant outer diameter portion having a constant outer diameter.
